(19) United States
(12) Patent Application Publication
(10) Pub. No.: US 2020/0345350 A1
(43) Pub. Date: Nov. 5, 2020

(54) SUCTION NEEDLE TRAP AND SURGICAL DRAPE

(71) Applicant: SHARP FLUIDICS LLC, Hayward, CA (US)

(72) Inventors: Josef E. GOREK, Ross, CA (US); Kenneth B. TRAUNER, San Francisco, CA (US); Douglas G. RIMER, Los Altos Hills, CA (US)

(73) Assignee: SHARP FLUIDICS LLC, Hayward, CA (US)

(21) Appl. No.: 16/855,787

(22) Filed: Apr. 22, 2020

Related U.S. Application Data
(63) Continuation of application No. PCT/US2018/059035, filed on Nov. 2, 2018.
(60) Provisional application No. 62/580,910, filed on Nov. 2, 2017.

(51) Int. Cl. A61B 17/06

(52) U.S. Cl. CPC A61B 17/06161; A61B 2017/00876

(57) ABSTRACT

Systems, devices and methods to improve safety and efficiency in an operating room may comprise needle receptacles configured for use with suction to store used needles. The devices can be safely worn for the surgeon to self-dispense new suture needles in the near surgical field and to secure the used needles into a needle receptacle with suction. Surgical drapes can also be configured to safely retain surgical instruments during a surgical procedure.

### CROSS-REFERENCE

This application is a continuation of International Patent Application No. PCT/US2018/059035, filed Nov. 2, 2018, published as WO 2019/090132 on May 9, 2019, which claims the benefit of the filing date of U.S. Patent Application No. 62/580,910, filed Nov. 2, 2017, entitled “Needle Trap”, the disclosures of which are incorporated, in their entirety, by this reference.

The subject matter of the present application is related to International Patent Application No. PCT/US2016/059599, filed Oct. 28, 2016, “Systems and Methods for Increased Operating Room Efficiency”, U.S. Provisional Application No. 62/248,029, filed on Oct. 29, 2015, entitled “Systems and Methods for Increased Operating Room Efficiency”, U.S. patent application Ser. No. 14/697,050, filed on Apr. 27, 2015, entitled “Systems and Methods for Increased Operating Room Efficiency”, and International Patent Application No. PCT/US2015/027659, filed Apr. 24, 2015, entitled “SYSTEMS AND METHODS FOR INCREASED OPERATING ROOM EFFICIENCY”, the entire contents of which are incorporated herein by reference.

### BACKGROUND

The use of an operating room can present expensive medical service costs. An operating room must be sterilized before each operation and the medical staff must also prepare for the operation. Because each employee is usually paid for their time in the operating room, the operating room use costs can be very high. It has been estimated that there are over one billion operating room passages of needles per year in the US, which can present a risk of injury. By increasing the efficiency of the employees within the operating room, the time for each procedure can be reduced and the cost of the surgery can also be reduced. Further, it is important to account for surgical objects such as needles and sponges during a surgical procedure. If a needle becomes lost during the surgery, steps need to be taken to ensure patient safety and that the needle has not been accidently left in the patient. Accounting for needles during a surgical procedure in an accurate manner can be time-consuming. Therefore, it would be desirable to provide improved ways to keep track of used needles in an operating room. Also, needle puncture through a surgical glove can present risks to operating room personnel.

The process of loading a needle holder is often carried out by those personnel assisting the surgeon in the process of surgery. A scrub technician or surgical assistant can pass the loaded needle holder to the surgeon. Both unused needles and used needles can be maintained on an instrument tray such as a Mayo stand, and an accounting of the needles is often made by the surgical assistant and circulating nurse during the course of surgery.

At the time of surgical incision wound closure, or other tissue repair, during which multiple armed sutures are to be utilized, the surgical assistant can be fully focused on the needs of the surgeon. The assistant passes the loaded needle holder to the surgeon's hand for use.

Used needles may be dispensed and accounted for in a less than optimal and safe manner. As a substitute for having the loaded needle driver passed to the surgeon, the surgeon may awkwardly load the armed suture himself. This often requires the surgeon turning to the instrument tray (e.g., Mayo stand), locating the suture package, and grasping and orienting the package such that the needle can be effectively and properly loaded onto the needle holder, which takes additional time and movement than would be ideal and undesirably directs the surgeon's attention away from the patient.

In the neutral zone approach to passing objects and instruction, the objects and instruments are passed between a scrub tech and a surgeon are placed in a neutral zone area, rather than being directly passed between scrub tech and surgeon. The process may require a scrub tech to place the object into the neutral zone and the surgeon cannot pick up the object until the scrub tech's hands are removed from the neutral zone. Similarly, when the surgeon no longer needs a surgical object, it is placed in the neutral zone and the surgeon's hand removed. This system can be less than ideal because the surgeon and scrub tech must often be very careful and clearly communicate and look at the neutral zone, away from the site of the operation, when any objects are passed. This can be particularly difficult when trying to perform actions quickly which can easily happen in an operating room procedure, for example when attempting to save a patient's life.

In many currently used suture handling methods and systems, the surgeon can be handed a needle driver with an armed suture needle. The surgeon may drive the needle through the flesh of the patient and then hands the needle driver with used needle to the scrub tech. The scrub tech then moves the used needle away from the surgical field and removes the used needle. The scrub tech then places a new armed needle in the needle driver and then hands the surgeon the needle driver. The described process is repeated, and results in more movement than would be ideal.

In addition to being highly inefficient, such systems can also have poor micro-ergonomics.

Similar challenges can occur with the management of surgical instruments such as electrocautery and suction devices. These devices are also passed between a surgeon and scrub tech during surgery. This can be a time consuming task and may result in more movement than would be ideal.

Although recent improvements such as surgical needle traps with needle driver slots and needle receptacles have been proposed, still further improvements or alternative approaches would be beneficial.

In light of the above, improved methods and apparatus are needed to improve operating rooms. Ideally, such methods and apparatus would provide at least some of the above mentioned deficiencies of the prior approaches and provide one or more of improved efficiency, outcomes, needle and instrument handling, counting, or safety.

### SUMMARY

The present invention relates to systems and methods for increasing operating room efficiency. Although specific reference is made to dispensing and securing needles and the temporary placement and retrieval of medical instruments such as electrocautery and suction devices, the embodiments described herein are well suited for use with many types of objects used in or outside an operating room.

Systems and methods for improving operating room efficiency as described herein improve the manner in which surgeons' access, storage, retrieve, and dispose of objects used in surgery such as sutures, needles, and surgical instruments. The methods and apparatus disclosed herein can improve safety by decreasing the number of needle and instrument passes between the surgeon and assistant, and by placing needles in a receptacle prior to being passed from the surgeon to the assistant or providing a system for temporarily placing or storing an instrument. The needle receptacle coupled to suction can facilitate the placement of the sharp object such as a needle in the needle receptacle. Also, the needle receptacle can be configured to decrease sound associated with surgical suction, such that medical personnel can readily communicate when the needle receptacle is being used. The suction can be configured such that the needle is placed in one or more of a landing zone or an entry zone, and then drawn into the secure zone when released from the needle driver. This approach can make it easier for the user to place the needle in the receptacle because the suction allows the use of a relatively large entry zone or landing zone and combinations thereof, so that user alignment with the needle receptacle is facilitated. This can allow the user such as a surgeon to focus on other tasks such as suturing the patient and needle reconciliation. The sound can be decreased in many ways, such as one or more of a streamlined flow path or a dampening structure to decrease one or more of turbulence or resonance.

Some embodiments relate to the secure retrieval and storage of surgical needles that can be facilitated and made more efficient and ergonomic by associating the needles, sutures, and instruments. The methods and apparatus disclosed herein allow the physician to self-load the needle into the needle driver, self-place the dispensed needle into a used needle receptacle, which have the benefits of decreasing reliance on assistants, improving operating room efficiency and the safety of needle handling. In some embodiments, one or more needles can be secured in the receptacle prior to passing the needle to an assistant, which increases safety by placing the needle in the receptacle prior to passing to the assistant. A plurality of needles can be surgeon dispensed and surgeon placed in the container, such that the safety and efficiency can be increased by decreasing the number of passes between the surgeon and assistant.

In a first aspect, a needle receptacle comprises: a lower structure having an entry zone; an upper structure coupled to the lower structure to at least partially define a secure zone therebetween; a needle slot for receiving one or more suture needles between the lower structure and the upper structure; and an outlet duct in fluid communication with the needle slot and configured to be in fluid communication with a suction source. In some embodiments, the needle receptacle further comprises: a filter located between the outlet duct and the needle slot. In some embodiments, the filter has: one or more apertures sized or shaped to prevent one or more needles from passing though the filter. In some embodiments, the needle receptacle further comprises: a transition zone between the entry zone and the secure zone. In some embodiments, the transition zone comprises a funnel structure between the entry zone and the transition zone. In some embodiments, the funnel structure comprises at least two opposing surfaces that are a first distance apart at or near the entry zone and a second distance apart at or near the secure zone, the second distance being less than the first distance. In some embodiments, the funnel structure is configured to increase an air flow from the suction source therethrough. In some embodiments, a first of the at least two opposing surfaces is coupled to the upper structure at the entry zone and a second of the at least two opposing surfaces is coupled to the lower structure at the entry zone. In some embodiments, the needle receptacle further comprises an adhesive coating on at least one of the at least two opposing surfaces to adhere to and securely retain one or more needles within the secure zone. In some embodiments, the needle receptacle further comprises one or more magnets at the secure zone to magnetically attract and securely retain one or more needles therewithin. In some embodiments, the needle receptacle further comprises a divider partitioning the secure zone into a first portion and a second portion, the first portion being located between the second portion and the entry zone, and the second portion being located between the first portion and the outlet duct. In some embodiments, the divider has: at least one aperture sized or shaped to allow passage of a needle from the first portion to the second portion. In some embodiments, the divided is sloped along a depth of the secure zone from a first end of the secure zone at the entry zone to an opposite second end of the secure zone opposite the entry zone. In some embodiments, one or more of the lower or upper structure is at least partially planar. In some embodiments, one or more of the lower or upper structure is at least partially sloped such that a plurality of needles is sorted by one or more of size, shape, or mass.

In another aspect, a needle receptacle comprises: a lower structure; an upper structure; a secure zone defined between the upper and lower structures, the secure zone comprising one or more dividers to divide the secure zone into a plurality of cavities; and an outlet duct in fluid communication with a suction source and the secure zone to apply suction toward the plurality of cavities to secure one or more needles therewithin, wherein the upper structure an opening therethrough to provide an entry zone into one or more cavities of the secure zone to allow the one or more needles to be deposited within said one or more cavities. In some embodiments, the upper structure is movable such that the opening is selectively positionable over at least one selected cavity of the plurality of the cavities, allowing deposition of the one or more needles into the at least one selected cavity. In some embodiments, the upper structure is rotatable. In some embodiments, at least one of the cavities comprises a blank cavity, wherein when the opening of the upper structure is positioned over the blank cavity, the remaining cavities are closed. In some embodiments, the opening is one or more of sized or shaped with a cross section having dimensions smaller than one or more of the size or shape of a cross section of one of the cavities. In some embodiments, the needle receptacle further comprises a side wall extending from a side of the lower structure toward the upper structure. In some embodiments, the side wall has one or more apertures to allow the outlet duct to be in fluid communication with the secure zone. In some embodiments, the lower structure has one or more apertures to allow the outlet duct to be in fluid communication with the secure zone.

In another aspect, a needle receptacle comprises: a lower structure having an outer perimeter; a side wall extending from the lower structure, the side wall and the lower structure defining a secure zone; an upper structure pivotably coupled to one or more of the lower structure or the side wall, the upper structure being positionable between a closed position covering the secure zone and an open position exposing the secure zone; an open cell foam structure within the secure zone, the open cell foam structure having an upper surface; and an outlet duct in fluid communication with the secure zone, the outlet duct being couplable in fluid communication with a suction source such that air can be pulled into the secure zone though the open cell foam structure and out the outlet duct, the air applying a holding force to one or more needles placed over the upper surface of the open cell foam structure. In some embodiments, the needle receptacle further comprises: a holding structure extending over at least a portion of the upper surface of the open cell foam structure to hold the open cell foam structure within the secure zone. In some embodiments, the holding structure comprises a grate or a screen. In some embodiments, the open cell foam structure is coupled to the lower structure or the walls with adhesive. In some embodiments, one or more of the upper or lower structures is planar. In some embodiments, the open cell foam structure is at least partially permeable. In some embodiments, the outlet duct is coupled to one or more of the lower structure or side wall.

In another aspect, a needle receptacle comprises: an upper structure and a lower structure forming a secure zone therebetween; an outlet duct formed at a first end of the secure zone; an entry zone formed though the upper structure at a second end of the secure zone, opposite the first end of the secure zone; and an air inlet formed between the upper structure and the lower structure; wherein the entry zone is between the air inlet and the secure zone. In some embodiments, the entry zone comprises an opening formed though the upper structure. In some embodiments, the needle receptacle further comprises: a first throat extending from the opening in the upper structure to accelerate air entering the needle receptacle and receiving needles entrained in the accelerating air. In some embodiments, the needle receptacle further comprises: a second throat formed between the upper structure and the lower structure, and being located between the opening and the air inlet to accelerate air entering the needle receptacle though the air inlet. In some embodiments, the needle receptacle further comprises one or more magnets on the lower structure at the secure zone to securely retain one or more needles within the secure zone. In some embodiments, the needle receptacle further comprises adhesive on the lower structure of the secure zone to securely retain needles within the secure zone. In some embodiments, the secure zone includes a first portion and a second portion, the first portion orientated at an angle with the second portion.

In another aspect, a needle receptacle comprises: a channel forming a secure zone for retaining one or more needles therein, the channel having a first end coupled to an outlet duct; a funnel forming a transition zone and in fluid communication the channel, a first end of the funnel coupled to the channel; an opening at a second end of the funnel, opposite the first end and configured to receive air and needles entrained within the air, therethrough. In some embodiments, the funnel has a first cross sectional area at the first end and a second cross sectional area at the second end, the first cross sectional area being greater than the second cross sectional area. In some embodiments, the needle receptacle further comprises one or more magnets or an adhesive in the secure area, between the transition zone and the outlet duct. In some embodiments, the needle receptacle further comprises an adhesive structure between the secure zone and the outlet duct to receive and securely retain needles thereon. In some embodiments, the adhesive structure comprises an air permeable membrane. In some embodiments, the adhesive structure is configured to translate relative to the needle receptacle to selectively expose one or more portions of the adhesive structure to the secure zone for receiving one or more needles on an exposed portion of the adhesive structure. In some embodiments, the needle receptacle further comprises: a needle chamber between the secure zone and the outlet duct to receive and securely retain needles therein.

In another aspect, a system for temporarily securely placing and retrieving surgical instruments during surgery comprises: a surgical drape; and a plurality of retention devices attachable to the surgical drape, the retention devices being configured to temporarily securely retain a surgical instrument placed over the retention devices. In some embodiments, the system further comprises: a plurality of complementary retention devices attachable to one or more surgical tools, the surgical tools comprising a lead and a tool piece. In some embodiments, the plurality of retention devices comprises one or more magnets. In some embodiments, the plurality is arranged on the surgical drape in a two-dimensional array. In some embodiments, an adhesive drape is adhered to the surgical drape, the adhesive drape having a perimeter, wherein one or more of the plurality of attachment devices are arranged along the perimeter of the adhesive drape. In some embodiments, each of the plurality of retention devices attachable to the drape imparts of retention force on the surgical instrument to securely hold the surgical instrument in place. In some embodiments, the surgical instrument is releasable from the attachment structure with leverage. In some embodiments, the surgical instrument is releasable from the attachment structure with a rotational movement of the surgical instrument. In some embodiments, the system further comprises: at least one lead connecting a tool price of the surgical instrument to a source of the surgical instrument; and at least one complementary attachment device at the at least one lead, the at least one complementary attachment device being engageable with the retention devices attachable to the drape.

In some embodiments, a method of tracking a medical instrument during a procedure in an operating room comprises: receiving the medical instrument on an entry zone of an instrument receptacle; directing the received medical instrument to a secure zone of the instrument receptacle from the entry zone with suction; and capturing the medical instrument within the secure zone. In some embodiments, the medical instrument comprises a suture needle. In some embodiments, directing the received medical instrument to the secure zone comprises coupling a vacuum source to a vacuum duct of the instrument receptacle, the vacuum duct being in fluid communication with the secure zone. In some embodiments, capturing the medical instrument within the secure zone comprises adhering the medical instrument to an adhesive structure. In some embodiments, the adhesive structure comprises a movable tape. In some embodiments, the method further comprises capturing a first surgical instrument on a first portion of the movable tape, moving the movable tape, and capturing a second surgical instrument on a second portion of the movable tape. In some embodiments, directing the received medical instrument to the secure zone comprises adhering the medical instrument to a magnetic structure. In some embodiments, the method further comprises allowing the captured medical instrument to visualized. In some embodiments, the instrument receptacle is at least partially translucent or transparent to allow the secure zone to be visualized. In some embodiments, directing the received medical structure to the secure zone comprises accelerating an air flow with a funnel structure disposed at or near a transition zone between the entry and secure zones of the instrument receptacle. In some embodiments, receiving the medical instrument on the entry zone comprises exposing the entry zone of the instrument receptacle. In some embodiments, exposing the entry zone of the instrument receptacle comprises opening a cover of the instrument receptacle. In some embodiments, exposing the entry zone of the instrument receptacle comprises moving a cover of the instrument receptacle such that an opening on the cover is over a selected portion of the secure zone.

In some embodiments, a needle receptacle comprises: a lower panel having an entry zone; a first side panel connected to the lower panel and foldable over the lower panel to at least partially define a secure zone therebetween; a needle slot for receiving one or more suture needles between the lower panel and the first side panel; and a suture pack holder extending from an edge of the lower panel at the entry zone. In some embodiments, the suture pack holder is coupled to the lower panel with a hinge. In some embodiments, the hinge comprises a living hinge. In some embodiments, the first side panel is connected to the lower panel at a first side of the lower panel. In some embodiments, the needle receptacle further comprises: a second side panel connected to the lower panel and disposed over the lower panel, wherein the secure zone is defined between the lower, first side, and second side panels, and wherein the second side panel is connected to the lower panel at a second side of the lower panel, the second side opposite the first side. In some embodiments, the needle receptacle further comprises: an upper needle driver slot between the first side panel and the second side panel and extending from the entry zone to the secure zone. In some embodiments, the needle receptacle further comprises: a lower needle driver slot through the lower structure and extending from the entry zone to the secure zone. In some embodiments, the needle receptacle further comprises: a transition zone between the entry zone and the secure zone; and a guide extending from the entry zone to the secure zone. In some embodiments, the guide is formed by the first side panel and a second side panel connected to the lower panel. In some embodiments, a first end of the guide is at the entry zone and a second end of the guide is at an upper needle driver slot that extends between the first side panel and a second side panel connected to the lower panel and extends from the entry zone to the secure zone. In some embodiments, one or more of the lower or first side panels comprise a transparent or translucent material.

In another aspect a kit comprises a needle trap and a suture pack. In some embodiments, the suture pack is attached to the suture pack holder. In some embodiments, the kit comprises a sterile kit.

In some embodiments, the air inlet comprises a streamlined profile along a flow path to reduce turbulence.

In some embodiments, a fluid path extending from the entry zone to the outlet duct comprises a streamlined profile to reduce turbulence.

In some embodiments, the streamlined profile comprises one or more of rounding edges or reduced tortuous fluid paths.

In some embodiments, fluid travels along the fluid path with a substantially laminar flow.

In some embodiments, fluid passing through the fluid path produces sound levels within a range from 65 decibels to 80 decibels.

In some embodiments, fluid passing through the fluid path produces sound levels less than 65 decibels.

In some embodiments, the needle receptacle is configured to draw a needle from the entry zone to the secure zone with a sound level within a range from 60 dB to 85 dB and a suction air flow within a range from 1 liter per minute to 150 liters per minute. In some embodiments, the needle is drawn from the entry zone to the secure zone along a trajectory transverse to a gravitational field. The earth's gravitational field may extend in a substantially vertical direction and the trajectory may extend in a substantially horizontal direction.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a perspective section view of a needle trap, in accordance with some embodiments.

FIG. 1B is a side section view of the needle trap of FIG. 1A.

FIG. 1C is a side section view of the secure zone of the needle trap of FIG. 1A.

FIG. 1D is a side section view of the transition zone of the needle trap of FIG. 1A.

FIG. 2A illustrates perspective section view of another needle trap, in accordance with some embodiments.

FIG. 2B illustrates a front view of the needle trap of FIG. 2A in an open configuration.

FIG. 2C illustrates a front view of the needle trap of FIG. 2B in a closed configuration.

FIG. 3A illustrates a perspective view of a rotary needle trap, in accordance with some embodiments.

FIG. 3B illustrates a top view of the rotary needle trap of FIG. 3A.

FIG. 3C illustrates a perspective view of another rotary needle trap, in accordance with some embodiments.

FIG. 3D illustrates a top view of the rotary needle trap of FIG. 3D.

FIG. 4 illustrates a side section view of an exemplary transition zone of the needle trap of FIG. 1A, in accordance with some embodiments.

FIG. 5 illustrates a side section view of another exemplary transition zone of the needle trap of FIG. 1A, in accordance with some embodiments.

FIG. 6A illustrates a perspective view of another needle trap, in accordance with some embodiments.

FIG. 6B illustrates a magnified perspective view of an embodiment of the secure zone of the needle trap of FIG. 6A.

FIG. 6C illustrates a magnified perspective view of another embodiment of the secure zone of the needle trap of FIG. 6A.

FIG. 7 illustrates a perspective view of a further needle trap, in accordance with some embodiments.

FIG. 8 illustrates a perspective view of a surgical drape system for placing and retrieving surgical instruments, in accordance with some embodiments.

### DETAILED DESCRIPTION

The present invention is directed towards systems and methods for improving the efficiency of operating rooms. The embodiments disclosed herein are well suited for combination with many prior systems and methods, such as prior suture packs, prior needle holders, prior surgical instruments, electrocautery, and prior operating rooms and personnel.

Although specific reference is made to the placement of used needles in a used needle container, the embodiments disclosed herein are well suited for use with needles dispensed from a suture pack and placed in a used needle container without suturing the patient, for example.

### Definitions

Secure—The needle is secure means that the tip of the needle is prevented from compromising sterility or coming into contact with skin of the patient or surgical staff. When used with the sharps container, the used needle is physically secured from falling out of container. Sharps can include needles and tools or other objects which have one or more sharp surfaces that can puncture the skin of the patient or surgical staff.

In many embodiments, a secure needle as described herein is secured to prevent both the leading and trailing ends or tips of the needle from coming into contact with skin, gloves, surgical apparel of the surgical staff, surgical drape, or patient.

As used herein like characters such as letters and numerals refer to like elements.

As disclosed herein, a used suture needle encompasses a suture needle dispensed from a suture pack.

As used herein the terms “needle driver” and “needle holder” are used interchangeably.

As used herein the terms “armed sutures” and “armed needles” are used interchangeably.

As used herein the terms “used needle holder”, “needle receptacle”, “used needle receptacle, “used suture needle receptacle”, “sharps container”, “needle trap”, and “needle receptacle means” are used interchangeably.

As used herein the terms “suture package”, “suture pack” and “suture package means” are used interchangeably.

As used herein “secure” means fixed or fastened so as not to give way, become loose, or be lost.

As used herein “innocuous” means incapable of contact with a human finger.

One approach for improving operating room efficiency is to reduce the dependence of the surgeon on the surgical assistant. For example, a surgical procedure can include performing a surgical procedure and then closing a patient's surgical incisions after the procedure is completed. The closing generally includes installing surgical sutures to hold the patient's body tissue together after the surgery. This surgical suture procedure can include needles loaded with sutures that are stored in a needle package and a needle driver. When needed, the surgeon uses a needle driver to grasp and remove a needle from the suture package. The needle point is pressed into the flesh, advanced along the trajectory of the needle's curve until it emerges and is pulled through. The trailing thread is then tied into a knot, such as a square knot or surgeon's knot. The size and shape of the needles may also vary depending upon the patient's needs.

In many embodiments, an “armed” suture comprises a suture that has a surgical needle attached. Furthermore, packages of armed sutures often contain more than one such suture and needle. The package may contain not only one, but also perhaps four, five and possibly more, such as 8 or more sutures and needles. In the course of surgery, many such armed sutures can often be used, each needing to be “loaded” onto the needle holder or “needle driver.” The surgeon can hold the needle driver in his dominant hand and a tissue forceps in the non-dominant hand in order to manipulate and hold tissues to be sutured. Thus, the surgeon can use both hands when suturing to self-dispense and self-secure the dispensed needles.

In some embodiments a system for improving efficiency eliminates the need for the assistant to provide needles to the surgeon when closing a patent's surgical wounds. Swaged needles with sutures may comprise a pre-packed eyeless needle attached to a specific length of suture thread. The suture manufacturer swages the suture thread to the eyeless atraumatic needle at the factory.

There are several shapes and sizes of surgical needles. These include: straight, ¼ circle, ⅜ circle, ½ circle, ⅝ circle, compound curve, half curved (also known as ski), half curved at both ends of a straight segment (also known as canoe), etc.

In some embodiments, operating room efficiency can be improved by allowing the surgeon to load suture needles to a needle driver. A surgeon may use a dominant hand to hold the needle driver and one or more suture packets can be attached to the non-dominant limb of the surgeon. The surgeon can then grasp the new suture needles from the suture packet on the non-dominant limb. For example, if the user is right handed, the surgeon may attach the suture package to the left arm or hand and use the right hand to handle a needle driver. The user can grasp a portion of a needle with the needle driver and remove the needle from the suture package. The user can then use the needle driver to press the needle point into the flesh of the patient. The needle is advanced along the trajectory of the needle's curve until it emerges from the flesh, and the needle and suture are pulled through. The trailing thread is then tied into a knot.

In some embodiments, a needle trap or needle receptacle as described herein is configured such that a user can slide a needle into the receptacle and have the needle be secured the moment the needle is released from the needle driver. The needle can be released using a single maneuver, and the needle can be immediately secured within the needle receptacle.

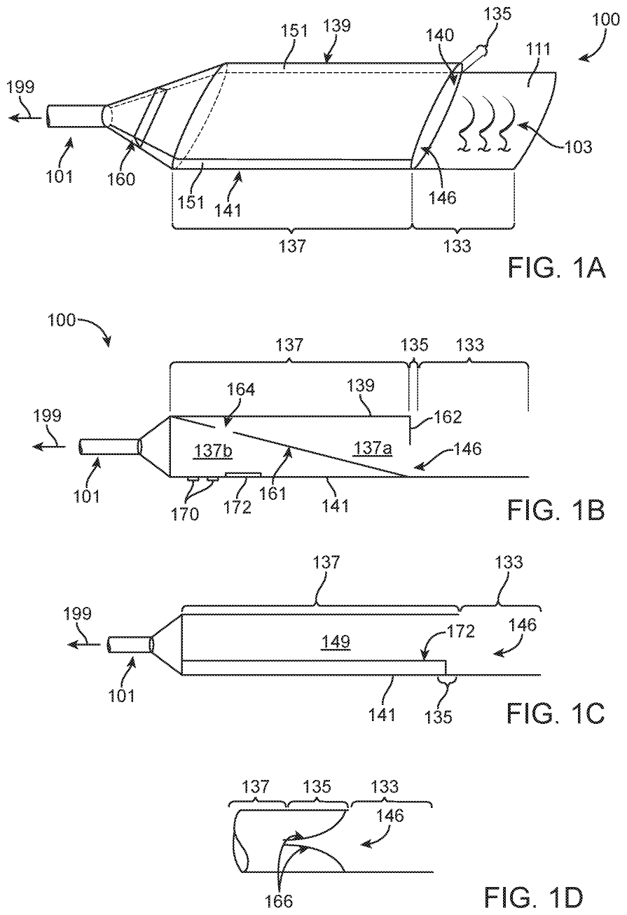

FIGS. 1A-1D illustrate a vacuum needle trap 100. The needle trap 100 may comprise a suction outlet duct 101 that is configured to be coupled to a vacuum or suction source. The needle trap 100 may further comprise a secure zone 137 having a needle slot 140, and the vacuum or suction source may be configured to be in fluid communication with the needle slot 140 via the suction outlet duct 101. The suction source may comprise any suction source available in many operating rooms. The needle trap 100 may further comprise an entryway or transition zone 135 leading to the secure zone 137. When suction or a vacuum is applied to the needle trap 100 via the vacuum or suction source, air may be pulled into the secure zone 137 and needle slot 140 through an opening 146 in the transition zone 135 from the vacuum outlet duct 101 (as shown by arrow 199).

The needle trap 100 may comprise a substantially planar device that comprises of one or more zones, such as one or more of the following zones: (1) a landing zone 133, (2) an entryway or transition zone 135, and (3) the secure zone 137. The needle trap may comprise each of these zones such that the needle trap comprises several zones. The needle trap 100 can include an upper structure 139 and a lower structure 141 that may be securely coupled together at their outer portions to define the interior and exterior of the needle trap 100. In some embodiments, the outer edges of the upper structure 139 and lower structure 141 are connected together directly. In some embodiments, the upper structure 139 and lower structure 141 are connected via side walls 151. Optionally, the needle trap 100 can include a needle driver slot extending through one or both of the upper structure 139 and a lower structure 141.

During use, a surgeon can use a needle driver to place and release a needle 103, with or without an attached suture, in the landing zone 133. The landing zone 133 may comprise a landing surface 111 that receives the needle. The needle 103 may then be entrained in the air flowing into the secure zone 137 of the needle trap 100. The moving air may impart force onto the needle 103 and the suture, causing the needle 103 and suture to move into the secure zone 137 of the needle trap 100.

The needle trap 100 may further comprise a filter 160. The filter 160 may be disposed before the vacuum outlet duct 101 near an end of the needle trap 100, for example, opposite of or distal to the landing zone 133. The filter 160 can be configured in many ways. The filter 160 may comprise channels sized smaller than the cross-section dimension of the needles 103 intended to be captured and secured in the needle trap 100, such that application of the vacuum pulls the needles 103 into the secure zone 137 without allowing the needles 103 and suture to pass through the filter 160, for example. The filter may comprise an open cell foam or HEPA filter, for example. The filter may comprise a screen, mesh, or other structure comprising a plurality of apertures extending therethrough. The filter 160 may allow fluids, such as air, to flow out of the needle trap 100 though the outlet duct 101. The filter 160 may aid in preventing solids, such as needles 103 and sutures and others, from exiting the needle trap 100 through the vacuum outlet duct 101. In some embodiments, the filter 160 may also aid in preventing liquids from flowing out of the needle trap 100. In some embodiments, the filter 160 comprises a screen with apertures smaller than the smallest diameter of a needle or suture used during surgery. In some embodiments, the needle trap 100 can be chosen for each surgery based on the size of needles used during surgery. For example, the needle trap 100 may be available in various filter channel sizes and may be labeled accordingly, such as on the upper structure 139, the lower structure 141, and/or the side walls 151.

In some embodiments, the filter 160 may comprise an adhesive surface that faces the secure zone 137 such that when the air pulls a needle 103 through the secure zone 137 and to the filter 160, the needle 103 may become adhered to the filter 160.

FIG. 1B shows a cross section of the needle trap 100. The secure zone 137 may comprise an opening 146 that may have a height that is less than the distance between the upper structure 139 and the lower structure 141, i.e., the depth of the secure zone 137. For example, one or more optional extensions such as walls 162 may extend from one or more of the upper structure 139 or the lower structure 141 to reduce the size of the opening 146 as compared to the height of the secure zone 137. Alternatively, the height of the secure zone 137 can be less than the height of the opening 146. For example, the secure zone 137 may be tapered from the opening 146 toward the vacuum outlet duct 101. In some embodiments, the needle trap 100 includes a divider 161 between a first portion 137a and second portion 137b of the secure zone 137. The divider 161 may comprise a sloped panel with one or more apertures 164 extending therethrough. The one or more apertures 164 can facilitate the flow of air though the divider panel 161.

In some embodiments, one or more of the apertures 164 may be sized and shaped to pass a needle 103 therethorugh. In such embodiments, the apertures 164 facilitate the flow of air and also the capture of needles 103. For example, as shown in FIG. 1B, a needle 103 may be drawn into the first portion 137a of the secure zone 137, then up the divider 161, then through one or more apertures 164, and then into the second portion 137b of the secure zone 137, where the needle 103 may be secured.

In some embodiments, the inner surfaces of the secure zone 137 may include adhesive 172 or magnets 170 to capture the needles 103. In such embodiments, once the needles 103 are drawn into the secure zone 137, they are securely held within the secure zone 137 via a magnetic force imparted on the needles by the magnets 170 or by adherence to the adhesive 172.

Alternatively, the divider 161 may comprise one or more apertures 164 sized smaller than the cross-sectional dimension of a needle, in order to deposit needles on the divider 161 itself. An adhesive can be deposited on the upper side of the divider 161 in order to secure the needles 103.

FIG. 1C shows a side, cross-section of the needle trap 100 comprising the adhesive surface. The surface of the lower structure 141 that faces the needle slot 140 of the secure zone 137 may comprise the adhesive 172, for example. In some embodiments, the adhesive 172 of FIG. 1C may be replaced with one or more magnets 170. In some embodiments, the magnets 170 may be located within the secure zone 137, either in addition to, or without the adhesive 172. The secure zone 137 may comprise an interior volume 149 through which air flows from the opening 146 to the outlet duct 101.

FIG. 1D shows an embodiment of the transition zone 135 that includes a funneled opening 146 between the landing zone 133 and the secure zone 137. The funneled opening 146 can aid in increasing the velocity of the air as it is pulled therethrough. The higher velocity air may aid in increasing the force of the air on entrained needles and facilitate pulling needles into the secure zone 137 with greater force as compared to the air flow without the funneled opening. The funnel may include two opposing surfaces 166 that are a first distance apart at or near the landing zone 133 and a second distance apart at or near the secure zone 137, the second distance being less than the first distance.

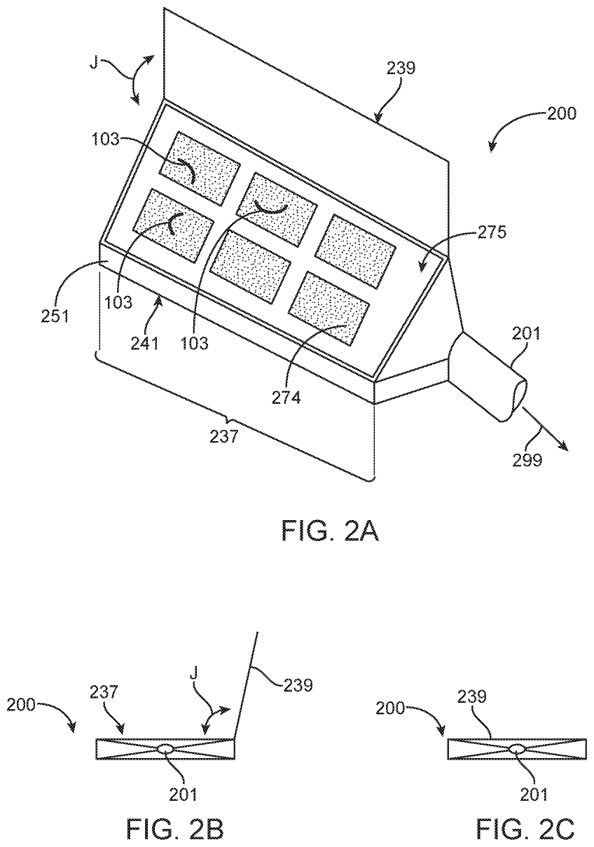

FIGS. 2A-2C illustrates a needle trap 200 that may be similar in at least some respects with the needle trap 100. The needle trap 200 may comprise a planar structure with a hinged upper structure 239 and a secure zone 237 in fluid communication with a suction outlet duct 201. The secure zone 237 may comprise a filter such as an open cell foam structure 274. The hinged upper structure 239 may be opened to expose the open cell foam structure 274 as shown in FIGS. 2A and 2B which shown the needle trap 200 in an open configuration. The open cell foam structure 274 may substantially fill the secure zone 237 of the needle trap 200. In some embodiments, the open cell foam structure may span the length and width of the secure zone 237, but may not fill the depth of the secure zone 237. The open cell foam structure 274 may be adhered or otherwise coupled to one or more of the bottom structure 241 or the sidewalls 251 of the needle trap 200. In some embodiments, the open cell foam structure 274 may be held in place within the secure zone 237 by a structure 275 that covers at least a portion of the top or exposed surface of the open cell foam structure 274. In some embodiments, the structure 275 may comprise a grate or a screen. Alternatively or in combination, the open cell foam structure 274 can be adhered to the bottom structure 241 of the needle trap 200. The suction outlet duct 201 may be configured to be connected to a vacuum or suction source for pulling air 299 out of the needle trap 200, particularly the secure zone 237, through the open cell foam 274.

During operation, air may be pulled though the top or exposed surface of the open cell foam structure 274 with enough velocity and force to pull and hold a needle 103 on the top or exposed surface. A surgeon may place the needle 103 on the upper surface of the foam structure 274 or release a needle 103 from a needle driver at or near the upper surface of the foam structure 274. The air flow through the foam structure 274 can then entrain the needle 103, pull the needle 103 towards the upper or exposed surface, and hold the needle 103 to the exposed surface of the foam 274.

After one or more needles 103 have been placed on the top or exposed surface of the foam structure 274, the hinged upper structure 239 may be closed over the top of the secure zone 237 and the open cell foam structure 274 to cover the needles 103 that are on the foam structure 274 and secure the needles 103 within the secure zone 237 of the needle trap 200, as shown in FIG. 2C. The upper structure 239 can be closed over the secure zone 237 (in the direction shown by double-sided arrow J) and secured with a latch, adhesive, Velcro®, or other mechanism to maintain the upper structure 239 in a closed configuration. The upper structure 239 may comprise a stiff structure connected to a housing of trap 200 with a hinge, for example, so as to allow the upper structure 239 to be folded over the foam to define the secure zone 237. The upper structure 239 may comprise an optically transmissive material to allow viewing of the needles contained therein, such as a transparent material. The needle trap 200 may comprise an optically transmissive filter in order to allow viewing of the needles with backlit illumination. For example, the housing of the needle trap 200 may comprise an optically transmissive material and the filter comprising foam 174 may comprise an optically scattering material for backlit illumination.

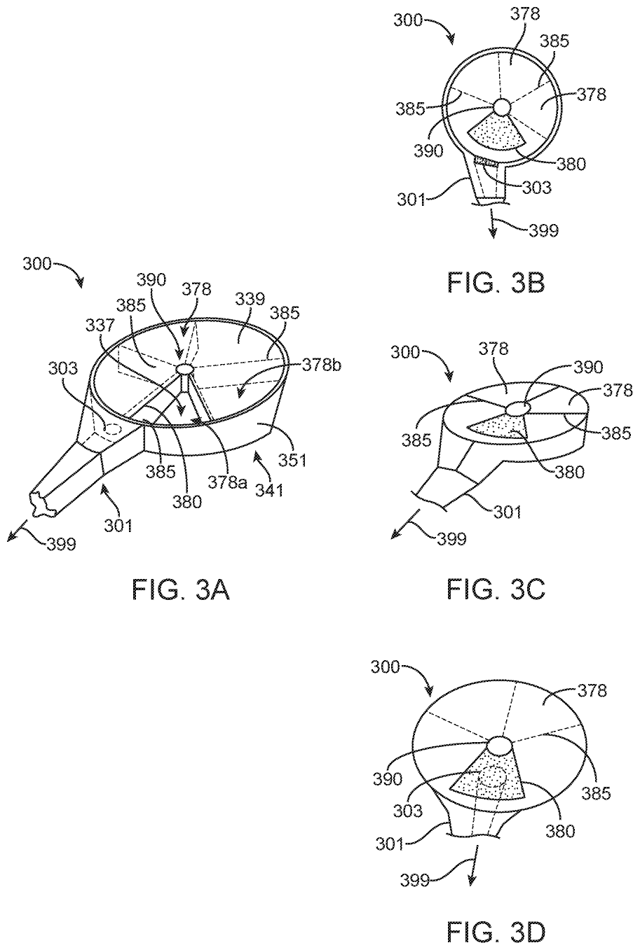

FIGS. 3A-3D illustrate a rotary needle trap 300 which may comprise a secure zone 337 for securely retaining one or more needles 103. The secure zone 337 may comprise one or more cavities 378 separated from each other by dividers 385 extending from an inner surface 381 of the lower structure 341 of the rotary needle trap 300 towards the upper structure 339 of the rotary needle trap. The dividers 385 also extend radially outward from a center 390 of the rotary needle trap 300. A first one or more of the cavities 378a may be configured to retain needles therein, such the cavity 378a while a second of the one or more cavities 378 may be a blank or empty cavity 378b. The needle trap 300 may also include an upper structure 339 that has a notch or other type of opening 380 that is sized and shaped with dimensions equal to or smaller than the size and shape of a cross section of a cavity 378. During use, the upper structure 339 may be rotated about its center 390 to selectively expose one of the cavities 378. When a selected cavity 378 is exposed or otherwise uncovered, a surgeon may place needles 103 within the selected cavity 378, e.g. 378a. Once a cavity 378a has the designated number of needles 103 placed within it, the upper structure 339 may be rotated to expose or open another cavity 378. When the desired cavities 378a are filled with needles 103, the cover may be rotated to an empty cavity 378b and the upper structure 339 may be secured in a position such that each of the cavities 378a for retaining needles 103 is covered to prevent release of the secured needles 103. An empty cavity 378b may comprise a portion of the needle trap 300 that is not configured to receive needles or may have a volume insufficient to securely retain needles therein. As with any of the upper structures of the needle traps described herein, the upper structure 339 may be at least partially transparent to facilitate viewing and counting of the captured needle(s) 103. Alternatively, the upper structure 339 may be fixed relative to the lower structure 341 and the opening 380 may be in a fixed location, and the secure zone 337 and its dividers 385 may be rotated about the center 390 by rotating a central knob, for example, to select a cavity 378.

In some embodiments, the needle trap 300 is fluidly coupled to a vacuum or suction source via a suction duct 301 to pull air out of the needle trap 300 (as shown by arrow 399) to help secure the needle(s) 103 within a selected compartment or cavity 378. The suction duct 301 may be in fluid communication with the secure zone 337 and its compartments or cavities 378 via one or more apertures 303. As shown in FIGS. 3A and 3B, the one or more apertures 303 may reside on the side wall 351 extending from the sides of the lower structure 341 toward the upper structure 339. As shown in FIGS. 3C and 3D, the one or more apertures 303 may, alternatively or in combination, reside on the lower structure 341. At least some of the dividers 385 may be porous to air flow such that vacuum or suction can reach the cavities 378 that are not immediately adjacent the one or more apertures 303. In some embodiments, one or more of the cavities 378 may include an open cell foam structure therein. As described above with respect to FIGS. 2A-2C, during use, air may be pulled though the open cell foam structure 274 to the upper or exposed surface of the open cell foam structure.

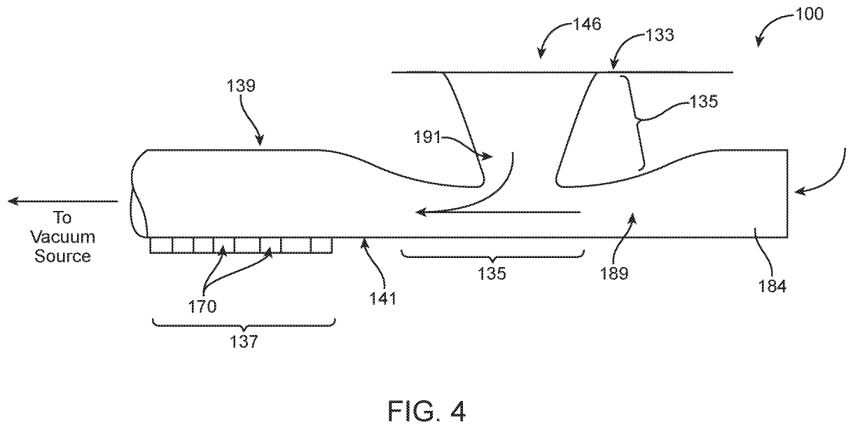

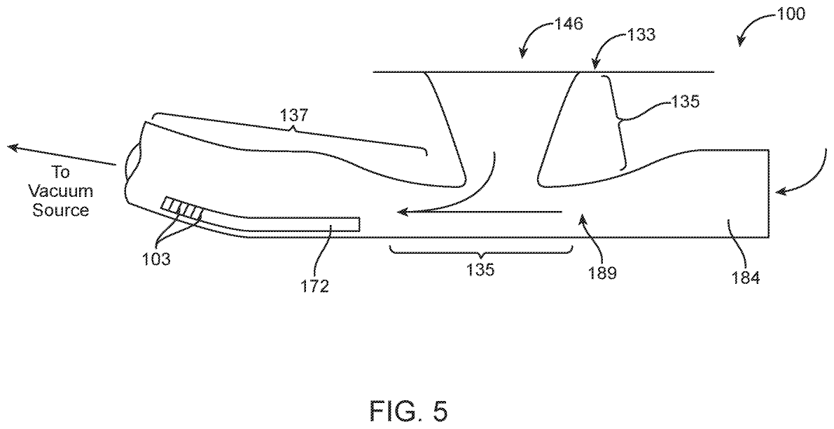

The needle traps disclosed herein may further comprise airflow features to increase the forces exerted by a vacuum or suction source on the needle(s) 103 and to facilitate retention of captured needle(s) 103. FIGS. 4 and 5 illustrate various airflow embodiments of the needle trap 100, for example. As described above, the needle trap 100 may comprise a landing zone 133, a transition zone 135, and a secure zone 137, which may be in fluid communication with a vacuum source.

During operation, air from the vacuum source may be pulled into the needle trap 100 via the air inlet 184 and the opening 146 in the landing zone 133. Air pulled in through the air inlet 184 may be accelerated in a throat 189 formed in the air inlet 184. The throat 189 may be located between the opening to the air inlet 184 and the transition zone 135, such that the air may be accelerated through the throat 189 before reaching the transition zone 135. Similarly, air entering through the landing zone 133 may be accelerated though a second throat 191 within the transition zone 135. The acceleration of the air through the throats 189, 191 can aid in increasing the force the air exerts on the needles 103 as they are pulled into and through the needle trap 100.

As shown in FIG. 4, the opening 146 may be formed though the upper structure 139 while the air inlet may be formed between the upper structure 139 and the lower structure 141.

During use, a surgeon may drop or place a used needle 103 though the opening 146 and into the landing zone 133 where air entering through the opening 146 may entrain the needle 103 and pull the needle 103 into the transition zone 135 where additional air that has entered the needle trap 100 through the air inlet 184 combines with the air from the opening 146 and carries the entrained needle 103 into the secure zone 137.

Once the needle 103 is within the secure zone 137 of the needle trap 100, the needle(s) 103 may be securely retained by either magnetic force imparted on the needle(s) 103 by magnets 170, for example, as shown in FIG. 4, or by adherence to adhesive 172, as shown in FIG. 5. In some embodiments, as shown in FIG. 5, the secure zone 137 comprises a first portion at an angle with a second portion. The angle between a central, longitudinal axis of each portion may be, for example, 5, 10, 15, 30, 45, or 60 degrees. The angled secure zone 137 may be aligned with the force of gravity such that needle(s) 103 within the secure zone 137 would flow against the force of gravity while the portion of the secure zone 137 closer to the vacuum source (e.g., the more downstream portion), may cause the needle(s) 103 to spread out along the secure zone 137 based on, for example, their mass, their resistance to air flow, or both.

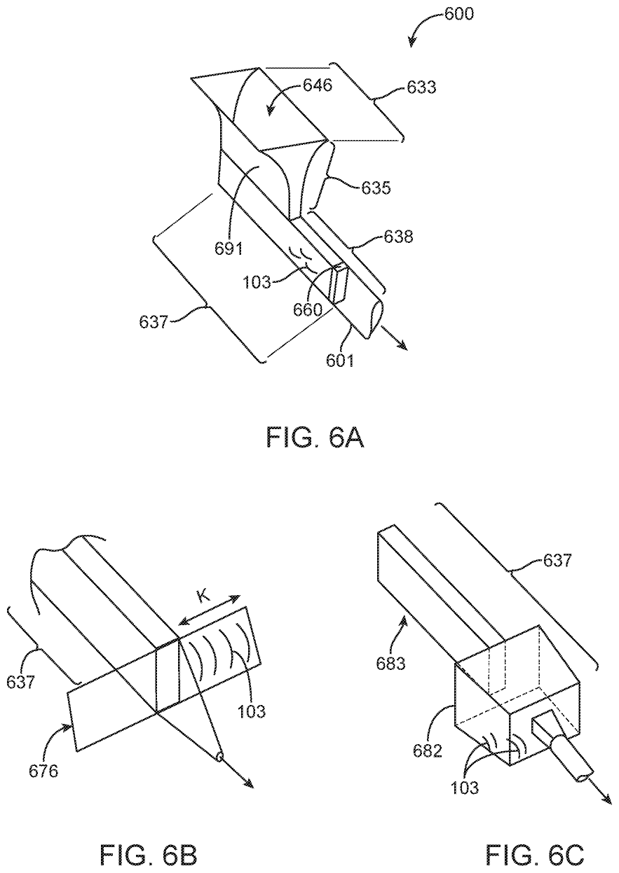

FIGS. 6A-6C illustrates a needle trap 600 having an opening 646 to receive the needle(s), entry zone or landing zone 633 and a transition zone 635 which may comprise a throat 691 which may be funnel shaped. The transition zone 635 may be fluidically coupled to an elongated secure zone 637. The secure zone 637 may be channel-shaped and have a first portion directly coupled to the throat 691 and a second portion 638 that may be located between the transition zone 635 and the outlet duct 601 of the needle trap 600. In some embodiments, a filter 660 is placed between the secure zone 637 and the duct 601.

During use, one or more needles 103 may be deposited into the entry zone 633 where air entrains the needle(s) 103 and pulls the needle 103 through the transition zone 635 and into the secure zone 637. As shown in FIG. 6A, the needles 103 may be retained within the second portion 638 of the secure zone. In some embodiments, the second portion 638 of the secure zone 637 includes adhesive and/or magnet(s) that retain the needle(s) 103 therein. In some embodiments, the secure zone 137 is at least partially translucent or transparent.

As shown in FIG. 6B, an air permeable adhesive structure 676, such as a stiff open cell foam card coated with adhesive, may be placed between the exit of the suction duct 601 and the secure zone 637. The adhesive structure 676 may act as a filter and may allow fluids, such as air, to flow out of the needle trap 600 though the outlet duct 601. The adhesive structure 676 may also aid in preventing solids, such as needle(s) 103 and sutures from exiting the needle trap 500. In some embodiments, the adhesive structure 676 also aids in preventing liquids from flowing out of the needle trap 600. In some embodiments, the adhesive structure 676 comprises a screen with apertures smaller than the smallest diameter of a needle 103 or suture used during surgery.

During operation, needle(s) 103 may be drawn towards the exit of the suction duct 601 by the air flowing through the secure zone 637. The needle(s) 103 may then contact and may be retained by the adhesive structure 676. Once a needle 103 is secured to a portion of the adhesive structure 676, the adhesive structure 676 may comprise an elongate tape and may be translated, for example, in a direction shown by arrow K, to bring a second portion of the adhesive structure 676 in proximity to the exit of the suction duct 601 such that a second exiting needle 103 may be retained thereon. Once the adhesive structure 676 has retained one or more needles 103, it may be removed from the needle trap 600 and the needle(s) 103 on the adhesive structure 676 may be reconciled and/or securely disposed.

As shown in FIG. 6C, a retention chamber 682 may be located in the secure zone 637, and coupled to the suction duct 601. The retention chamber 682 may have a volume and/or a cross-sectional area (taken in a plane perpendicular to the air flow) that is greater than that of the channel 683 of secure zone 637. By increasing the volume and/or the cross-sectional area, the velocity of the air flowing through the retention chamber 682 can be reduced as compared to the velocity of the air flowing through the channel 183. In some embodiments, the flow is reduced such that the air does not impart a substantial force onto the needle(s) 103 and pull them into the outlet duct 601. Instead, upon entering the retention chamber 682, the air velocity may be reduced and the needle(s) may fall to the bottom of the retention chamber 682, where a magnet or adhesive may be located. In some embodiments, a filter is placed between the suction duct 601 and the retention chamber 682 to aid in retaining the needle(s) 103 within the retention chamber 682.

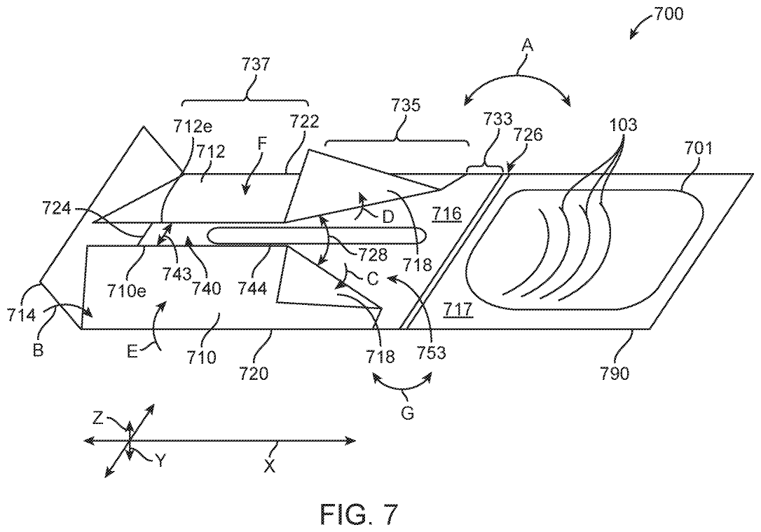

FIG. 7 illustrates another needle trap 700 that may have features in common with various needle traps 700 described herein. The needle trap 700 is shown in a partially assembled configuration alongside suture pack holder 790, which can hold a suture pack 701. The needle trap 700 may be made from a single sheet of material. For example, as shown in FIG. 7, the body of the needle trap 700 may be formed from a single, folded sheet of material. The needle trap 700 can comprise a substantially planar device that comprises several zones: (1) a landing zone 733, (2) an entry zone or transition zone 735, and (3) a secure zone 737.

The secure zone 737 of the needle trap 700 may be formed by folding panels 710, 712, and 714 over a top of a bottom panel 716. For example, the end panel 714 may be folded over the top of the bottom panel 716 of the needle trap 700 in a direction indicated by arrow B and along fold 724, which may comprise a hinge. Similarly, side panels 710, 712 may be folded over the top of the bottom panel 716 of the needle trap 700 in a direction indicated by arrows E and F, respectively, and along folds 720, 722, respectively, which may comprise hinges.

The folded panels 710, 712, 714 and bottom panel 716 may form a needle slot 740 for securely storing used suture needles, such as suture needles 103. The panels 710, 712, 714, 716 may be affixed or otherwise coupled together in a folded position using, for example, an adhesive. In some embodiments, the panels 710, 712, 714, and 716 are sonically welded or thermally bonded in a folded position. The needle slot 740 may comprise a cross-sectional length defined by the distance between folds 720 and 722, and a variable height defined by the distance between the upper panel 710 and the bottom panel 716 and the upper panel 712 and the lower panel 716, for example.

The needle trap 700 may include a needle driver slit or slot 743 extending between the edges 710e, 112e, of the side panels 710, 712, respectively, in the folded configuration. In some embodiments, one side panel extends over another side panel such that the needle driver slot 743 is defined by the separation between edges 710e and 712e, respectively, as the needle driver tip advances a used needle along the slit into the secure zone. In some embodiments, a needle driver slot 744 may be formed though the bottom panel 716. The needle driver slots 743, 744 may be configured to provide clearance for the needle driver along the entire length of the needle translation from the landing zone 733 to the secure zone 737.

The configuration of the needle trap 700 can be described with reference to an elongate axis X that extends from left to right in FIG. 7, along a length of the needle trap 700, and a transverse axis Y that extends up and down in FIG. 1, along a width of the needle trap 700 when viewing the front or top of the needle trap 100 from the perspective of the surgeon, and a depth axis Z which defines a depth.

In some embodiments, the landing zone 733 can be a flat zone or area formed by a portion of the bottom panel 716 that extends beyond the transition zone 735 and away from the secure zone 737. The landing zone 733 may be an exposed part of the bottom panel 716 that is not covered by the side panels 710, 712. The surgeon can hold the used needle(s) 103 with a needle driver and place them on an upper surface of the landing zone 733. The contact and/or force of the needle 103 against the landing zone 733 can cause the curvature of the used needle(s) 103 to be moved into a planar orientation flat against the landing zone 733 with the convex mid-portion of the curved needle 103 facing or pointing towards the transitional zone 735. The needle driver and needle 103 may then move along the elongate axis X into the needle slot 740, wherein the needle 103 is securely retained.

The needle driver slot 744 can extend into the landing zone 733 and the width of the needle driver slot 744 can be greater or oversized in the landing zone 733 to facilitate fast location of the entrance to the needle trap 700 with the needle driver. The needle driver slot 744 can taper as it extends through the transition zone 735 towards the secure zone 737, to provide a self-centering close fit with the tip of the needle driver in the transition zone 735 and secure zone 737.

The transition zone 735 may be disposed between the landing zone 733 and the secure zone 737. In the transition zone 735, the compressive side load on the needle ends, for example, as provided by the side panels 710, 712 and the bottom panel 716, may be increased and the depth axis of the needle slot 740 can be narrowed as the secured needle(s) 103 are translated through the transition zone 735, constraining the needle(s) 103 to a single needle deep array extending longitudinally along the secure zone 737.

The transition zone 735 may include a concave, wedge, or “V” shaped, guide 753 with the apex of the “V” shape pointing towards the secure zone 737 to promote proper orientation of the needles 103 and guide the needles and the needle driver towards the secure zone 137. The guide 753 may be formed by folding guide panels 718 at an angle, for example, a 15, 30, 45, 60, or 75 degree angle with the elongate axis X along folds 728. The panels 718 may be folded upwards and over the top of the side panels 710, 712, away from the bottom panel 716, as indicated by arrows C and D, or downwards and underneath the side panels 710, 712, and towards the bottom panel 716, in a direction opposite those indicated by arrows C and D. In some embodiments, the side panels 710, 712 may be cut along at an angle for example, a 15, 30, 45, 60, or 75 degree angle to form the guide.

In a preferred embodiment, the needle 103 is moved into contact with the landing zone 133 of the lower panel 716 by the surgeon manipulating the tip of the needle driver in the needle driver slot 744. The needle(s) 103 can be pushed against the landing zone 733 and become aligned with the plane of the bottom panel 716 of the used needle trap 700. The needles 703 can then be moved in translation along the elongate axis of the used needle trap 700 from the landing zone 733 into the transition zone 735 where the needles 703 slide into the used needle slot 740 with the convex side facing the secure zone 737 and the sharp tip and tail of the needle 103 facing the landing zone 733. The needle driver can move the used needle(s) 703 into the used needle slot 740 in the secure zone 737 until the needle driver runs into the end of the needle slot 740 or the last inserted used needle 703.

The needle trap 700 may include a suture pack holder 790. The suture pack holder 790 may be a panel 717 of the same sheet of material from which the rest of the needle trap 700 is made. In such an embodiment, the panel 717 may extend directly from the lower panel 716. In some embodiments, a hinge 726 may be located between the lower panel and the panel 717. The hinge 726 may be a living hinge, fold, crease, weakened portion of the material, or other type of hinge. In some embodiments, the suture pack holder 790 may rotate about the hinge 726 between an open position (as shown in FIG. 7) and a closed position, wherein, for example, the panel 717 is folded over the top of the bottom panel and one or more of the other panels 710, 712, 714. In some embodiments, the panel 717 may be folded beneath the bottom panel 716, in the direction shown by arrow G.

In some embodiments, the material from which the needle holder is made may be translucent or transparent such that the needles within the secure zone may be counted by a member of the surgical team to reconcile the needles. In some embodiments, the one or more of the panels 710, 712, 714, 716, 717 may be translucent or transparent.

Each of the embodiments described herein can be configured to decrease sound in one or more of many ways. In some embodiments, the needle receptacle comprises a streamlined fluid flow path to substantially decrease turbulence of fluid passing through the needle receptacle, so as to decrease noise associated with the suction. For example, a fluid path beginning at the landing zone 133, continuing through the transition zone 135 and the secure zone 137, and exiting through suction outlet duct 101 may comprise a streamlined profile with streamlined features, such as rounded corners, smooth size transitions, and straightened flow paths to substantially decrease fluid turbulence. Decreasing turbulence through the use of a streamlined fluid path may result in decreased noise and optionally an increase in laminar flow to reduce noise. Alternatively or in combination, the needle receptacle may comprise acoustic one or more dampening structures to decrease noise, such as one or more of foam, open cell foam, soft foam that absorbs acoustic waves, screens that can decrease resonance along the flow path and combinations thereof. These dampening structures can be located along an interior of the needle receptacle, such as along an interior of the entry zone or the secure zone. For example, the acoustic dampening structure can be provided at locations corresponding to the open cell foam as described herein. Alternatively or in combination, the acoustic dampening structure can be placed at locations along the flow path to decrease resonance, such as near filter 160 as shown in FIG. 1A, and the filter 160 may comprise an acoustic dampening structure to inhibit acoustic resonance along an elongate axis of the needle receptacle extending from the entry zone to the outlet duct as described herein, for example. Alternatively, the acoustic dampening structure can be located on an upper surface of the interior of the needle receptacle, such as above the adhesive 172 and on an upper side of the interior of the needle receptacle with reference to FIG. 1C, for example.

The needle receptacles as described herein can be configured to draw a needle from one or more of a landing zone or an entry zone into a secure zone of the needle receptacle with a flow rate within a range from about 1 liter per minute (l/min) to about 200 liters per minute (l/min), within a range from about 2 l/min to about 100 l/min, or within a range from about 10 l/min to about 100 l/min. The flow rate may comprise 1 l/min, 10 l/min, 20 l/min, 50 l/min, 100 l/min, 200 l/min, and can be within a range defined by and two of the preceding values. The corresponding acoustic sound can be within a range from about 60 decibels (dB) to about 85 dB, within a range from about 65 dB to about 80 dB. The acoustic sound may comprise 60 dB, 65 dB, 70 dB, 75 dB, 80 dB or 85 dB and can be within a range defined by any two of the preceding values. The corresponding acoustic sound may comprise no more than about 65 dB, for example.

In some embodiments, the needle receptacle is configured to draw the needle from the entry zone as described herein to a secure zone as described herein with a sound level within a range as described herein, e.g. from 60 dB to 85 dB, and a suction air flow within a range as described herein, e.g. from 1 liter per minute to 150 liters per minute. For example, the needle can be drawn from the entry zone to the secure zone along a trajectory transverse to a gravitational field of the earth, for example at an angle of about 45 degrees to the gravitational field of the earth. As one of ordinary skill in the art will appreciate, the earth's gravitational field extends in a substantially vertical direction. In some embodiments, the trajectory extends along a substantially horizontal direction.

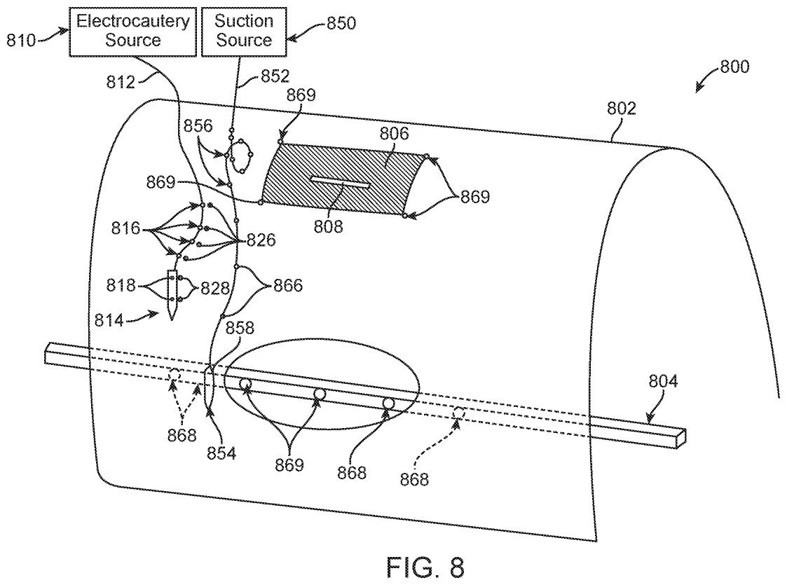

FIG. 8 shows the system 800, for safely and securely storing and retrieving instruments, such as electrocautery and suction devices, during surgery. As discussed above, in the neutral zone approach to handling objects and instruments, these items are passed between a scrub tech and a surgeon and are placed in a neutral zone area as part of the passing process. The process may require a scrub tech to place the object into the neutral zone and the surgeon cannot pick up the object until the scrub tech's hands are removed from the neutral zone. Similarly, when the surgeon no longer needs a surgical object, it is placed in the neutral zone and the surgeon's hand removed. This system is less than ideal because the surgeon and scrub tech must often be very careful and clearly communicate and look at the neutral zone, away from the site of the operation, when any objects are passed. This can be particularly difficult when trying to perform actions quickly which can easily happen in an operating room procedure, for example when attempting to save a patient's life.

Even when dealing with less risky passing procedures, such as passing a suction device into and out of the surgeon's hands, the neutral zone approach takes the surgeon's attention away from the surgical site in order to complete a safe pass.

The system 800 may include one or more retention devices 826, 828, 866, 868, 869, such as magnets, that are attached to a surgical drape 802 that is placed over the patient during surgery or they may be attached to a rail 804 of a surgical bed or other surgical platform. The surgical instruments may include complementary attachment devices 816, 818, 856, such as magnets, or may have inherent retention properties that are complementary to the retention devices 826, 828, 866, 868, 869. For example, in some embodiments, the instruments include a ferrous metal that is attracted to magnets, but not include or be a magnet. In some embodiments, the instruments include both a complementary retention device and also have properties that are complementary to the retention devices.

FIG. 8 shows two examples of surgical instruments that may be used with the system 800, an electrocautery system 810 and a suction system 850. Each system 810, 850 may include a lead. For example, electrocautery system includes an electrical cable leads 812 that connects the electrocautery source with the electrocautery tool piece 814 and suction system includes a tube lead 852 that fluidically and mechanically couples the suction source to the suction tool 854.

The attachment devices for the tool pieces 814, 854 and the leads 812, 852 may differ. For example, the attachment devices 816, 856 for the leads may impart a low attachment force on the corresponding lead through the corresponding attachment devices 816, 818 such that more than one set of engaged attachment devices may be required to support the weight of the tool pieces 814, 854. Such a configuration may allow a surgeon to pick up the tool pieces 814, 854 and move it around and separate the attached lead 812, 852, from the attachment devices with minimal force. In some embodiments, the attachment force between the lead and the attachment devices may be such that the lead may be easily unzipped from the drape 802 without substantially disturbing the drape 802.

The tool pieces 814, 816 may attach to one or more attachment devices with sufficient attachment force to securely hold the tool piece in a particular position. For example, in a position in which the surgeon placed the tool piece for temporary storage during a surgery.

In some embodiments, the attachment devices 818 are located on the tool piece 814, 854 such that a surgeon may leverage the tool piece to separate the tool piece from the engaged attached devices. For example, with an elongated tool piece, such as the electrocautery device, may include a single attachment device 818 at a distal end of the tool piece. Such a placement may allow a surgeon to retrieve the tool piece by griping and lifting the tool piece at an opposite end, such as a proximal end of the tool piece, to lever the tool piece and disengage the attachment devices.

In some embodiments, the tool piece includes one or more attachment devices 818 at or near a central location of the tool piece. Such a placement may allow a surgeon to retrieve the tool piece by griping and rotating the tool piece about its center while lifting the tool piece to disengage the attachment devices.

In some embodiments, the retention devices 826, 828, 866, 868, 869 on the drape 802 may be arranged in a two-dimensional array with a spacing between adjacent devices that matches the spacing between attachment devices 816, 818, 856, 858 located along the leads and the tool pieces 814, 854.

In some embodiments, the drape 802 may include an adhesive drape zone 806 in a location proximate to and surrounding an incision site 808. The adhesive drape zone 806 may include an adhesive drape that adheres to the patient's skin. In some embodiments, the adhesive drape is incorporated into the drape 802, while in other embodiments the adhesive drape is applied to the drape 802 and/or the patient's skin to couple the drape 802 to the patient. The adhesive drape of the adhesive drape zone 806 may also reinforce the skin and aids in preventing tearing of the incision 808.

The drape 802 may also include attachment devices located at or near the adhesive drape zone 806. For example, in the embodiment shown in FIG. 8 attachment devices 869 are located along the perimeter of the adhesive drape zone 806. Attachment devices 869 located near the adhesive drape zone 806 may allow a surgeon to temporarily securely place a tool piece 814, 854 within the near surgical field. By securely placing the tool piece 814, 854 in the near surgical field, the surgical team can avoid having to pass the tool piece into and out of the near surgical field. This can reduce the workload on the surgical team and may prevent dropping of the tools during otherwise unnecessary passing procedures.

In some embodiments, the attachment devices may be embedded within the drape 802. For example, in embodiments with two layer drapes, the attachment devices may be secured between the layers of the drape. In some embodiments, the attachment devises are partially coated with an adhesive such that they can be applied to a surface of the drape 802 in a location determined by the surgical team.

In some embodiments, the system may also include attachment devices on a bed rail 804 of a surgical bed. For example, the bed rail 804 includes six attachment devices 868. Similar to the other attachment devices, the attachment devices 868 may be sized and shaped to securely hold a tool piece 814, 854. The attachment devices 868 may be arranged in an array along the bed rail 804 to provide multiple secure locations for the surgeon to place the tool piece.

The systems and methods as described herein may be used to safely handle sutures and surgical devices during procedures involving non-living subjects, such as during the performance of autopsies on cadavers, wherein the person operating on the subject may still be exposed to blood-borne pathogens. Alternatively or in combination, the systems and methods described herein may be used to safely handle sutures and surgical devices during procedures involving non-human subjects, such as during the performance of an operation of an animal (e.g., in a veterinary practice or in animal studies).

Each of the needle receptacles as described herein may be provided in a sterile kit. The sterile kit may comprise a sterile barrier material defining a sterile package in which the needle receptacle is contained prior to the sterile package being opened and the sterile needle receptacle removed for use as described herein.

While preferred embodiments of the present invention have been shown and described herein, it will be obvious to those skilled in the art that such embodiments are provided by way of example only. Numerous variations, changes, and substitutions will now be apparent to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A needle receptacle comprising:
a lower structure having an entry zone;
an upper structure coupled to the lower structure to at least partially define a secure zone therebetween;
a needle slot for receiving one or more suture needles between the lower structure and the upper structure; and
an outlet duct in fluid communication with the needle slot and configured to be in fluid communication with a suction source.

2. The needle receptacle of claim 1, further comprising:
a filter located between the outlet duct and the needle slot.

3. The needle receptacle of claim 2, wherein the filter has:
one or more apertures sized or shaped to prevent one or more needles from passing though the filter.

4. The needle receptacle of claim 2, further comprising:
a transition zone between the entry zone and the secure zone.

5. The needle receptacle of claim 4, wherein the transition zone comprises a funnel structure between the entry zone and the transition zone.

6. The needle receptacle of claim 5, wherein the funnel structure comprises at least two opposing surfaces that are a first distance apart at or near the entry zone and a second distance apart at or near the secure zone, the second distance being less than the first distance.

7. The needle receptacle of claim 6, wherein the funnel structure is configured to increase an air flow from the suction source therethrough.

8. The needle receptacle of claim 6, wherein a first of the at least two opposing surfaces is coupled to the upper structure at the entry zone and a second of the at least two opposing surfaces is coupled to the lower structure at the entry zone.

9. The needle receptacle of claim 6, further comprising an adhesive coating on at least one of the at least two opposing surfaces to adhere to and securely retain one or more needles within the secure zone.

10. The needle receptacle of claim 1, further comprising one or more magnets at the secure zone to magnetically attract and securely retain one or more needles therewithin.

11. The needle receptacle of claim 1, further comprising a divider partitioning the secure zone into a first portion and a second portion, the first portion being located between the second portion and the entry zone, and the second portion being located between the first portion and the outlet duct.

12. The needle receptacle of claim 11, wherein the divider has:
at least one aperture sized or shaped to allow passage of a needle from the first portion to the second portion.

13. The needle receptacle of claim 12, wherein the divided is sloped along a depth of the secure zone from a first end of the secure zone at the entry zone to an opposite second end of the secure zone opposite the entry zone.

14. The needle receptacle of claim 1, wherein one or more of the lower or upper structure is at least partially planar.

15. The needle receptacle of claim 1, wherein one or more of the lower or upper structure is at least partially sloped such that a plurality of needles is sorted by one or more of size, shape, or mass.

16. A needle receptacle comprising:
a lower structure;
an upper structure;
a secure zone defined between the upper and lower structures, the secure zone comprising one or more dividers to divide the secure zone into a plurality of cavities; and
an outlet duct in fluid communication with a suction source and the secure zone to apply suction toward the plurality of cavities to secure one or more needles therewithin,
wherein the upper structure an opening therethrough to provide an entry zone into one or more cavities of the secure zone to allow the one or more needles to be deposited within said one or more cavities.

17. The needle receptacle of claim 16, wherein the upper structure is movable such that the opening is selectively positionable over at least one selected cavity of the plurality of the cavities, allowing deposition of the one or more needles into the at least one selected cavity.

18. The needle receptacle of claim 17, wherein the upper structure is rotatable.

19. The needle receptacle of claim 16, wherein at least one of the cavities comprises a blank cavity, wherein when the opening of the upper structure is positioned over the blank cavity, the remaining cavities are closed.

20. The needle receptacle of claim 16, wherein the opening is one or more of sized or shaped with a cross section having dimensions smaller than one or more of the size or shape of a cross section of one of the cavities.

21. The needle receptacle of claim 16, further comprising a side wall extending from a side of the lower structure toward the upper structure.

22. The needle receptacle of claim 21, wherein the side wall has one or more apertures to allow the outlet duct to be in fluid communication with the secure zone.

23. The needle receptacle of claim 16, wherein the lower structure has one or more apertures to allow the outlet duct to be in fluid communication with the secure zone.

24.-86. (canceled)
